(19) United States
(12) Patent Application Publication
(10) Pub. No.: US 2024/0245394 A1
(43) Pub. Date: Jul. 25, 2024

(54) SPINAL RETRACTOR BLADE AND RELATED RETRACTOR DEVICE AND METHOD

(71) Applicant: James B Carr, Atascadero, CA (US)

(72) Inventor: James B Carr, Atascadero, CA (US)

(21) Appl. No.: 18/563,219

(22) PCT Filed: May 26, 2021
(86) PCT No.: PCT/US21/34246

§ 371 Date: Nov. 21, 2023

(51) Int. Cl. A61B 17/02; A61B 17/56; A61B 17/70; A61F 2/44; A61F 2/46

(52) U.S. Cl. CPC A61B 17/0206; A61B 17/7074; A61F 2/4455; A61F 2/4611; A61B 2017/0256; A61B 2017/564; A61F 2002/4628

(57) ABSTRACT

A spinal retractor blade and retractor device are provided. The retractor blade may include an optional straight first leg having an end joined to an end of a shank. The blade further includes a transition arcuately extending from the straight first leg by about 90° to 180° and terminating in a free end. A width of the transition is suitable for receiving a spinous process. Retractors in accordance with the invention may optionally include a pair of such blades, and the blades themselves may optionally include a second straight leg between the transition and the free end.

### I. BACKGROUND OF THE INVENTION

### A. Field of Invention

The invention generally relates to the field of spinal retractors.

### B. Description of the Related Art

Surgical retractors have been known in various forms for many years. Naturally, there is no one retractor that is suitable for every procedure or every patient. Generally, retractors are designed to retract specific kinds of tissue or organs so as to expose a surgical field that is suitable for a specific procedure. More specifically, various specialized retractor blades are known for retracting specific kinds of tissue. For example, a retractor specifically adapted to retract skin is not necessarily well adapted to retract vertebrae. In certain procedures such as non-fusion decompression laminectomy interlaminar stabilization and Transforaminal Lumbar Interbody Fusion (TLIF) it is necessary to precisely size the gap between adjacent vertebrae to provide room for inserting a stabilizing device. Known retractors are not suitable for engaging the vertebrae e.g., through the spinous processes. Manufacturers of such non-fusion and fusion stabilizing devices do not provide spacing tools or retraction devices. The problem with these devices is that the vertebrae tend to move when the spacer and/or surgical retractors currently utilized are removed. Thus, the surgeon must repeatedly make adjustments and check the spacing prior to implanting the device. This lengthens the procedure and increases the chances that the patient may experience a complication like a dural leak, dural tear, nerve root injury, or infection. What is missing in the field is a retractor that is specifically designed for reliably engaging the vertebrae, spreading them to a predetermined degree, and holding them in position prior to inserting the stabilizing implant. Some embodiments of the present invention may provide one or more benefits or advantages over the prior art.

### II. SUMMARY OF THE INVENTION

Some embodiments may relate to a retractor blade comprising a straight first leg having an end joined to an end of a shank, wherein the straight first leg is from 0.01 cm to 6.0 cm in length. The blade further comprises a straight second leg having a free end, the straight second leg oriented parallel to the straight first leg, wherein the straight second leg is from 0.01 cm to 6.0 cm in length; and, a transition from the straight first leg to the straight second leg, wherein a width between the straight first leg and the straight second leg is operable to receive a spinous process.

Embodiments may relate to a retractor blade, comprising: a straight first leg having an end joined to an end of a shank; and a transition arcuately extending from the straight first leg between about 90° to 180° and terminating in a free end, wherein a distance between the straight first leg and the free end is suitable for receiving a spinous process.

Embodiments may relate to a spinal retractor, comprising: a first blade comprising: a straight first leg having an end joined to an end of a first shank; a straight second leg having a free end, the straight second leg oriented parallel to the straight first leg; and a transition from the straight first leg to the straight second leg, wherein a distance between the straight first leg and the straight second leg is suitable for receiving a spinous process; a second shank opposing the first shank; a shank spreader in spreadable communication with the first shank and the second shank; and a ratchet in ratcheting communication with the shank spreader.

Embodiments may relate to a method of vertebral retraction comprising the steps of: (1) providing a first vertebra and a second vertebra; (2) providing a spinal retractor, comprising a first blade. The first blade comprising: a straight first leg having an end joined to an end of a first shank; a straight second leg having a free end, the straight second leg oriented parallel to the straight first leg; and a transition from the straight first leg to the straight second leg, wherein a distance between the straight first leg and the straight second leg is suitable for receiving a spinous process; a second shank opposing the first shank; a shank spreader in spreadable communication with the first shank and the second shank; and a ratchet in ratcheting communication with the shank spreader; inserting first blade between two adjacent vertebrae; actuating the shank spreader, causing the first blade to impinge a spinous process of the first vertebra; and separating the first vertebra from the second vertebra by a predetermined amount.

Other benefits and advantages will become apparent to those skilled in the art to which it pertains upon reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, wherein like reference numerals indicate like structure, and wherein:

FIG. 1 perspective view of a retractor blade embodiment incorporated in a retractor;

FIG. 2A is a top view of an embodiment having a straight second leg terminating along a center line of a shank;

FIG. 2B is a top view of an embodiment having a straight second leg somewhat longer than that of FIG. 2A;

FIG. 2C is a top view of an embodiment having a straight second leg somewhat longer than that of FIG. 2B;

FIG. 3A is a top view of an embodiment having no straight second leg and having a 180° full transition;

FIG. 3B is a top view of an embodiment having no straight second leg and having a 135° partial transition;

FIG. 3C is a top view of an embodiment having no straight second leg and having a 90° partial transition;

FIG. 3D is a top view of an embodiment having not straight first or second leg;

FIG. 4 is a partial view of a retractor embodiment having a pair of blades according to an embodiment of the invention;

FIG. 5 is a front perspective view of a scissor-type retractor embodiment having a pair of blades according to an embodiment of the invention;

FIG. 6 is a magnified partial view of the portion of FIG. 5 enclosed in box 6;

FIG. 7 is a front elevation view of a rack-and-pinion style retractor embodiment;

FIG. 8 is a downward view of a rack-and-pinion embodiment cooperating with a spine; and

FIG. 9 is a perspective view of a rack and pinion embodiment having a folding handle.

### IV. DETAILED DESCRIPTION OF THE INVENTION

As used herein the terms “embodiment”, “embodiments”, “some embodiments”, “other embodiments” and so on are not exclusive of one another. Except where there is an explicit statement to the contrary, all descriptions of the features and elements of the various embodiments disclosed herein may be combined in all operable combinations thereof.

Language used herein to describe process steps may include words such as “then” which suggest an order of operations; however, one skilled in the art will appreciate that the use of such terms is often a matter of convenience and does not necessarily limit the process being described to a particular order of steps.

Conjunctions and combinations of conjunctions (e.g. “and/or”) are used herein when reciting elements and characteristics of embodiments; however, unless specifically stated to the contrary or required by context, “and”, “or” and “and/or” are interchangeable and do not require every element of a list or only one element of a list to the exclusion of others.

Terms of degree, terms of approximation, and/or subjective terms may be used herein to describe certain features or elements of the invention. In each case sufficient disclosure is provided to inform the person having ordinary skill in the art in accordance with the written description requirement and the definiteness requirement of 35 U.S.C. 112.

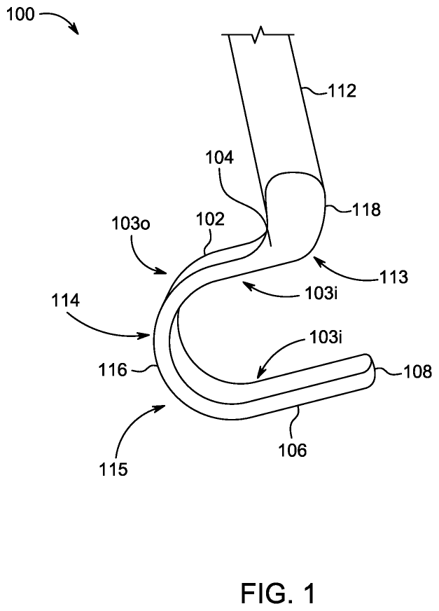

Referring now to the drawings wherein the showings are for purposes of illustrating embodiments of the invention only and not for purposes of limiting the same, FIG. 1 illustrates a retractor blade 115 embodiment 100. As described in more detail herein, retractor blades are often, but not necessarily, incorporated into spinal retractor embodiments in pairs. Thus, while the embodiment of FIG. 1 is described simply as a “retractor blade 115”, the very same structure is referred to elsewhere herein as a “first retractor blade 115f”. Though the structure is the same, the appended letter “f” is used to distinguish it from a “second retractor blade 115s”.

With continuing reference to FIG. 1, the retractor blade 115 includes an optional straight first leg 102 having an end 104 that is joined to the end 118 of a shank 112 of a retractor. The rest of the retractor is out of view. In the illustrated embodiment 100 the end 104 of the retractor blade 115 is joined to the shank 112 through a welding process; however, in this context the word joined is intended to include any known means for connecting the blade 115 to the shank 112 of a retractor. For example, and without limitation, the word joined includes the blade and shank being cast from a common mold, the blade and shank being formed from a common rod e.g., by bending the rod to a desired shape, or the blade being fastened to the shank as with a rivet or other known fastener.

Regardless of how the blade 115 is joined to the shank 112, it is desirable, though not required, to position the shank 112 on an outside face 1030 of the blade 115. Thus, a face 113 of an end 118 the shank 112 may be joined to the end 104 of the blade. Equivalently, a face 113 of an end 118 the shank 112 may be joined to an outside face 1030 of the blade 115 near the end 104.

FIG. 1 also shows an optional straight second leg 106 connected to the optional straight first leg 102 through a transition 114. The transition 114 may be arcuate, as shown, and thus may have an apex 116. The specific morphology of the illustrated transition 114 is a circular arc; however, the person having ordinary skill in the art will readily understand that the invention is not limited to circular arcs or arcuate morphologies in general. It will be understood by the ordinarily skilled artisan that the legs 102, 106 comprise the straight portions of the blade 115, as shown in FIG. 1, and the transition 114 comprises the balance of the blade 115. The straight second leg 106 terminates in a free end 108, and the straight second leg 106 is oriented parallel the straight first leg 102.

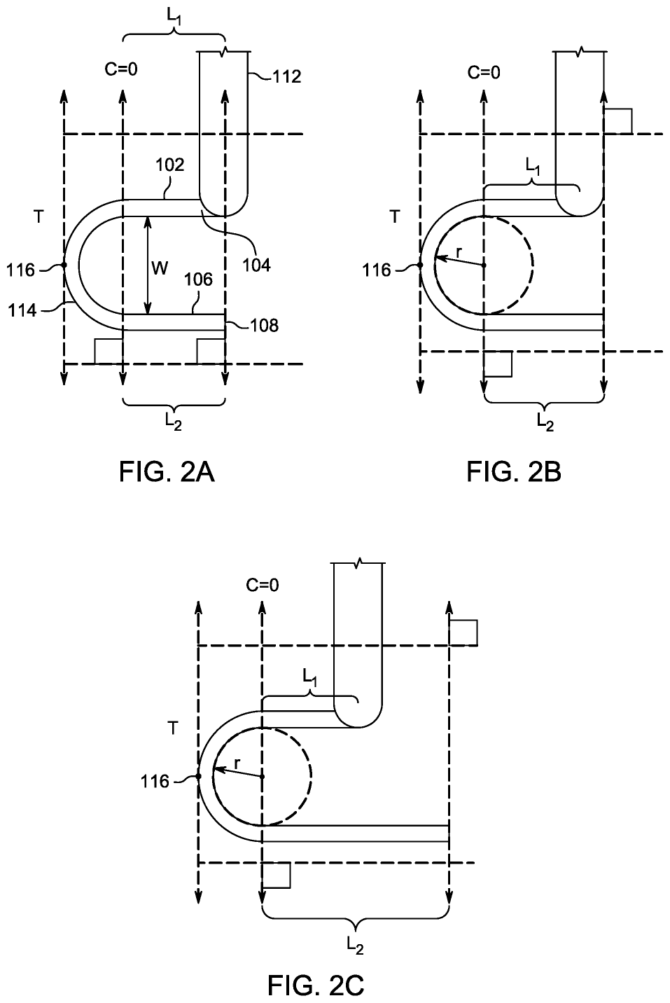

FIG. 2A a top view of a blade embodiment where the spacing between the straight first leg 102 and the straight second leg 106 is indicated by letter “w”. The spacing w can vary substantially from one embodiment to another because an operable spacing depends on the size of the spinous process with which it is to cooperate. Spinous processes vary in size from one patient to another, and from one vertebra to another within the same patient. The person having ordinary skill in the art will understand that the fit between the blade 115 and spinous process is not critical. Thus, a given blade size may be operable in connection with varying sizes of spinous processes; however, the governing principle is that the blade must be large enough to receive the spinous process yet small enough to fit between two adjacent spinous processes without unduly interfering with the surgeon's view or surrounding tissues. This principle provides parameters within which the person having ordinary skill in the art can determine operable dimensions without undue experimentation. However, operable dimensional ranges include a width w from about 0.3 cm to 2.0 cm+/−10%; the length (L₁, L₂) of a straight first leg 102 or straight second leg 106 may be from zero to 6.0 cm+/−10%; and, the height h of the blade as described in more detail subsequently herein may be from about 0.3 cm to 4.0 cm+/−10%.

With continuing reference to FIG. 2A, the length of the straight first leg 102 is denoted L₁. L₁ is measured from an end (line C=0) of the transition 114 to the end 104 of the region where the straight first leg 102 is joined to shank 112. Similarly, the length of the straight second leg 106 is denoted by L₂. L₂ is measured from an end (line C=0) of transition 114 to the free end 108 of the straight second leg 106. The ends of the transition 114 are indicated by the line C=0 which is the point at which the curvature of the blade becomes zero. Stated equivalently, the length of the straight second leg 106 is determined by the length L₂ of the blade exceeding the distance between a tangent line T of an apex 116 of the transition 114 and a parallel line drawn through an end of the straight second leg 106 most proximal to the tangent line T. As shown in FIGS. 2B and 2C, the straight second leg 106 can extend an arbitrary distance beyond the centerline of the shank 112, constrained only by the requirement that the blade must fit between two spinous processes without unduly interfering with the surgeon's view or surrounding tissues. FIGS. 2B and 2C also illustrate that the embodiment shown therein comprises a circular arc transition having a radius r and an apex 116 at 90° between the first and second straight legs 102, 106. Further, the transition 114 can be defined as the region of the blade falling between line C=0 and the tangent line T through apex 116, where lines C=0 and T are parallel to each other.

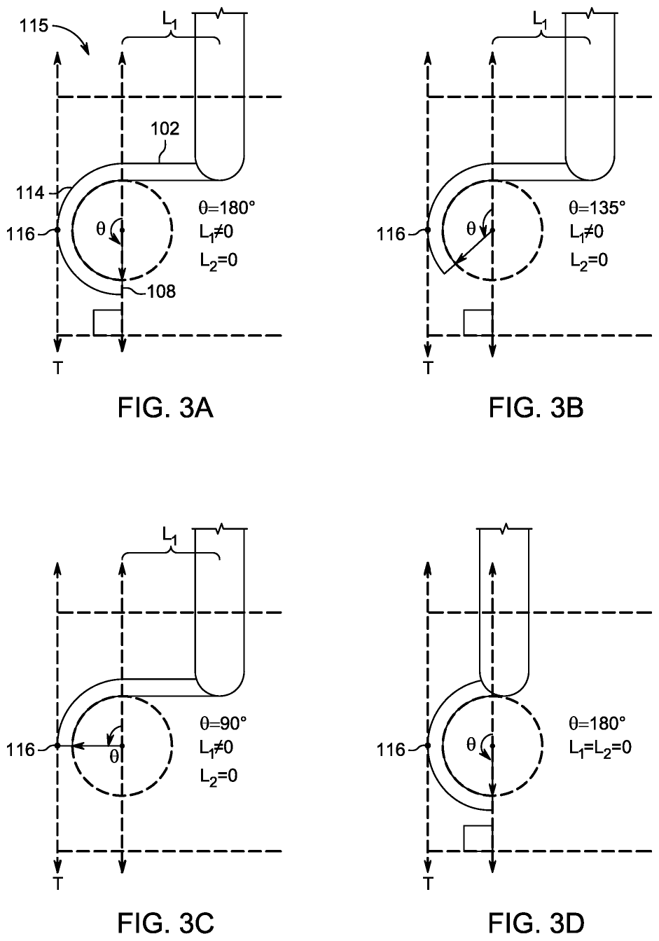

The lengths L₁, L₂ of the straight first and second legs 102, 106 can vary from one embodiment to another according to the anatomy of the patient; however, the upper limit of the lengths is constrained by the legs' interference with adjacent tissues and vertebrae. In other words, if the legs 102, 106 are too long they will impinge upon adjacent vertebrae and potentially cause tissue damage or interfere with the fit or use of the device. There is no lower limit to the length of the legs 102, 106. The lengths L₁, L₂ may either or both be zero, thus leaving only the transition. However, it may be advantageous to include legs 102 and/or 106 to better stabilize the device and improve its grip of the spinous processes. However, as described further in reference to FIGS. 3A-3D, embodiments are contemplated, and claimed herein, where straight first and/or second legs 102, 106 are absent.

FIGS. 3A through 3C illustrate embodiments lacking a straight second leg 106 (L₂=0), but having a straight first leg (L₁≠0). Though the straight second leg 106 may be advantageous in certain embodiments, it is not a requirement. The necessary cooperation between a blade 115 according to the invention and a spinous process can be achieved with less material. For example, and without limitation, in FIG. 3A a θ=180° transition 114 is provided but the blade 115 terminates 108 without extending into a straight second leg. Similarly, in FIG. 3B a partial transition of θ=135° is provided, and in FIG. 3C a partial transition of θ=90° is provided. Again, these drawings are not intended to limit the invention to a particular length(s) of transition 114. Rather, they are intended to illustrate that any length can be suitable, even a length of θ=90°, provided that the blade can separate vertebrae without sliding off the spinous processes. FIG. 3D illustrates an embodiment where both the straight first leg 102 and the straight second leg 106 are absent, that is where L₁=L₂=0.

Table I shows dimensions of spinous processes of male and female L1 to L5 vertebrae.

| | | Mean Size (mm) | | |
|---|---|---|---|---|
| Spinous Process | Size Range (mm) | Male | Female | SD |

Height

| | | | | |
|---|---|---|---|---|
| L1 | 16-36 | 26 | 23 | 4 |
| L2 | 18-35 | 27 | 24 | 3 |
| L3 | 18-38 | 27 | 24 | 3 |
| L4 | 14-32 | 24 | 21 | 3 |
| L5 | 10-34 | 20 | 18 | 4 |

Width

| | | | | |
|---|---|---|---|---|
| L1 | 6-16 | 11 | 10 | 2 |
| L2 | 6-16 | 11 | 10 | 2 |
| L3 | 5-17 | 11 | 10 | 2 |
| L4 | 4-18 | 9 | 8 | 2 |
| L5 | 3-15 | 9 | 8 | 2 |

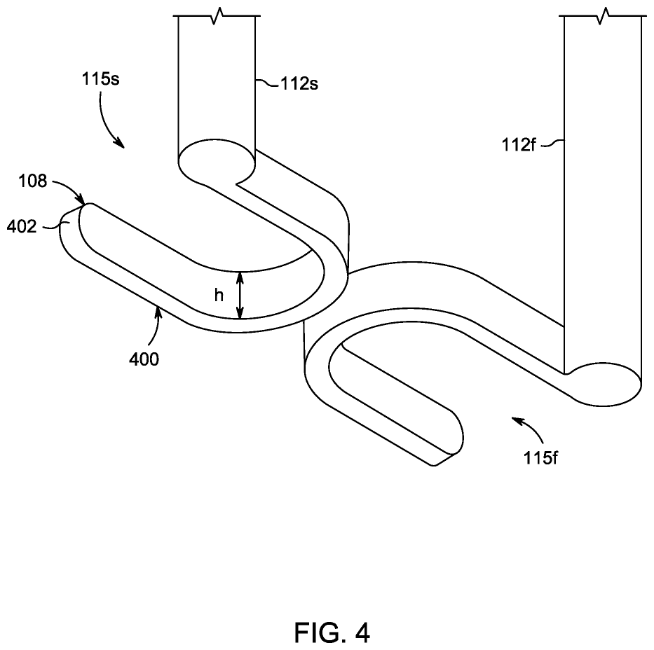

Turning to FIG. 4, a pair of opposing blades 115f, 115s joined to opposing shanks 112f, 112s are shown. The blades 115f, 115s comprise a flat band 400 of material having arcuate free ends 108 defining apexes 402. The shape of the free end may advantageously, but not necessarily, be arcuate. An advantage to an arcuate end 108 is that it tends to prevent or limit damage to surrounding tissues. The height h of the band 400 is not critical; however, it must cooperate with a spinous process. Thus, its height h is constrained by the blade's ability to fit between adjacent spinous processes without unduly interfering with or damaging the surrounding tissue. For example, the person having ordinary skill in the art will understand that a certain amount of force must be applied to adjacent spinous processes to separate them. Thus, a blade having an excessively small height h would apply too much force to a small area, unnecessarily cutting into the tissue. At the other extreme, an excessively large height h would add more material to the blades without adding more contact area with the spinous processes. The foregoing explanation provides parameters within which the person having ordinary skill in the art can determine operable dimensions without undue experimentation.

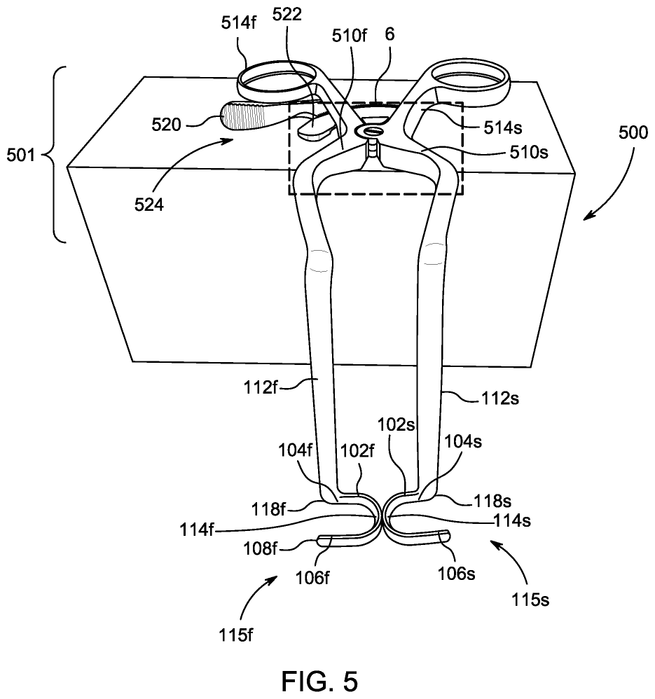

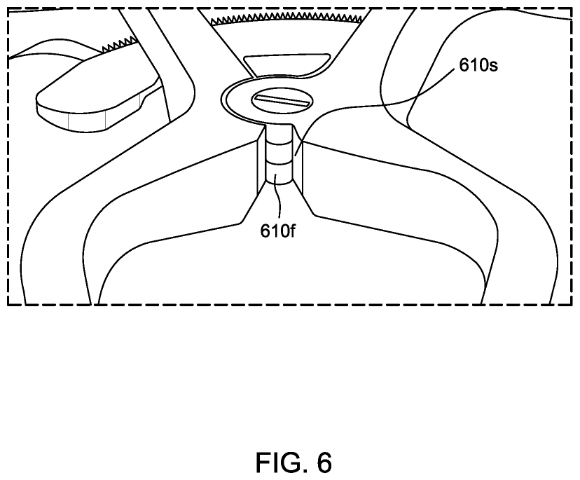

FIG. 5 is a front elevation view of a scissor-style embodiment 500. The shank spreader 501 has a first member 510f and a second member 510s. The first member 510f has a first handle 514f and a first shank 112f. Similarly, the second member 510s has a second handle 514s and a second shank 112s. The shank spreader 501 further includes the structure contained in box 6 of FIG. 5. This structure can be seen in greater detail in FIG. 6, which is a magnified partial view of the embodiment 500 in FIG. 5. The first and second members 510f, 510s are shown pivotably joined through the first pivot joint 610f and the second pivot joint 610s according to any suitable known means.

With continuing reference to FIG. 5, the shank spreader 501 further includes a ratchet 524 comprising an arcuate gear strip 522 and a spring-loaded pawl handle 520. The pawl handle 520 is biased toward the gear strip 522, therefore, the ratchet is disengaged by pulling back on the pawl handle 520.

According to the embodiment 500 of FIG. 5, the retractor includes shanks 112f, 112s having a 90° bend, which prevents the surgeon's hands from obstructing his view of the surgical field. The 90° bend is an advantageous feature but not a requirement of the invention. A first blade 115f is disposed at an end 118f of the first shank 112f. Similarly, a second blade 115s is disposed at an end 118s of the first shank 112s. The blades 115f, 115s are the same structure previously described in reference to FIG. 1. Accordingly, both have an optional straight first leg 102f, 102s; a transition 114f, 114fs; an optional straight second leg 106f, 106s; an end 104f, 104s joined to the end 118f, 118s of opposing shanks 112f, 112s; and a free end 108f, 108s.

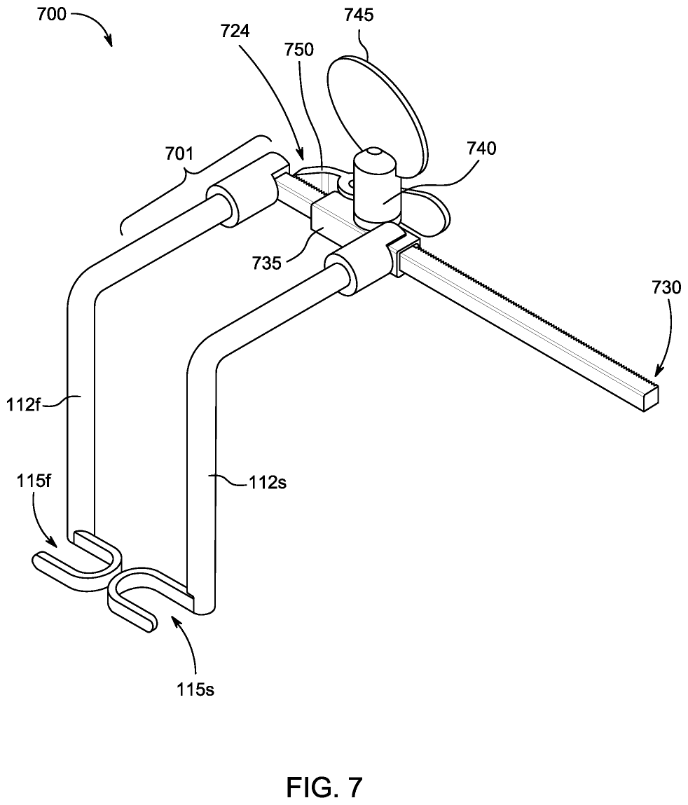

FIG. 7 is an illustration of a rack-and-pinion style retractor embodiment 700. The shank spreader 701 of this embodiment comprises a rack 730 fixedly joined according to any suitable known means to a first shank 112f. As used herein the term fixedly joined does not limit the invention to particular design choices, but rather is intended to be broadly construed to include any structure or structures that fix the orientation of one member to another, even including unitary parts made from a common mold. The gear teeth of the rack 730 are facing into the page out of view. A carrier 735 is fixedly joined to the second shank 112s and slideably engages the rack. The carrier 735 includes a knob 740 and handle 745 that rotatably communicates with a pinion gear contained within the carrier 735 and out of view. The pinion gear engages the teeth of the rack 730. Therefore, turning the knob 740 clockwise moves the carrier 735 linearly along the rack 730, thereby spreading the blades 115f, 115s apart. The carrier also includes a ratchet 724 mounted thereto. The ratchet 724 includes a pawl 750 which engages the teeth of the rack 730 such that it allows the carrier 735 to spread the blades 115f, 115s but must be disengaged to bring the blades back together.

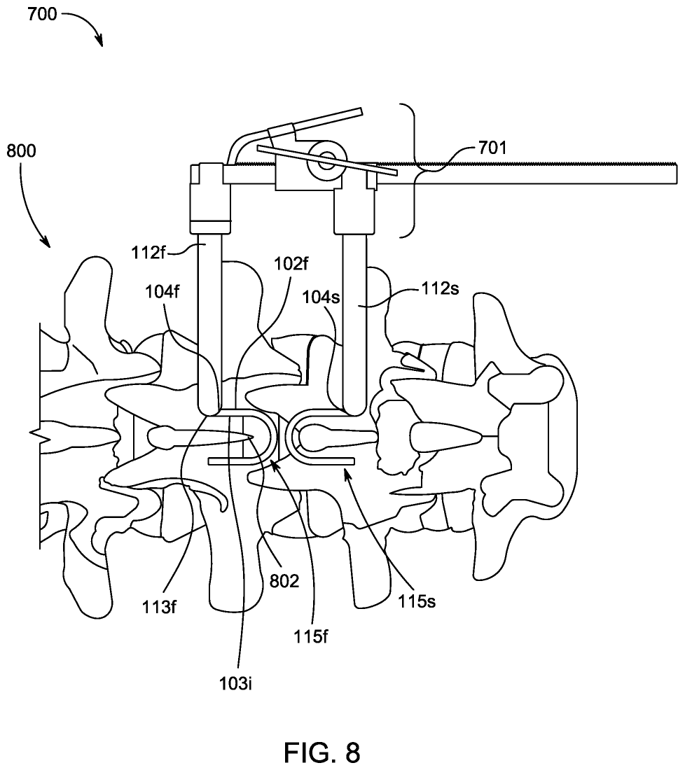

FIG. 8 illustrates the rack-and-pinion style retractor embodiment 700 of FIG. 7 in cooperation with a spinal column 800 from the point of view of the surgeon. As shown, a face 113f of the shank 112f does not extend beyond an inside face 103i of the straight first leg 102f. This arrangement of the shanks 112f, 112s at the ends 104f, 104s of the blades 115f, 115s keeps the shanks 112f, 112s clear of the surgical field and thus provides the surgeon with an unobstructed view. For convenience of illustration, the blades 115f, 115s appear to float near the spinous processes 802; however, in practice, the blades would abut the spinous processes thus providing a clear view of the surgical field.

The view shown in FIG. 8 is a top perspective with the retractor embodiment 700 positioned away from the viewer. This is typical of what a surgeon would see during use of an embodiment 700. The shanks 112f, 112s extend away from the user, placing the shank spreader 701 well outside the surgical field.

Placing the shank spreader opposite the surgeon prevents the embodiment 700 from obstructing the surgeon's view and keeps the embodiment 700 out of the way of the surgeon's hands. FIG. 8 further illustrates an advantage of placing the shanks 112f, 112s at the ends 104f, 104s of the blades 115f, 115s. Namely, doing so places the shanks as far as possible from the surgeon's view of the surgical field. Another advantage is that the shanks 112f, 112s themselves function to longitudinally distract soft tissue which tends to eliminate the need for additional retractors, and thus further declutters the field.

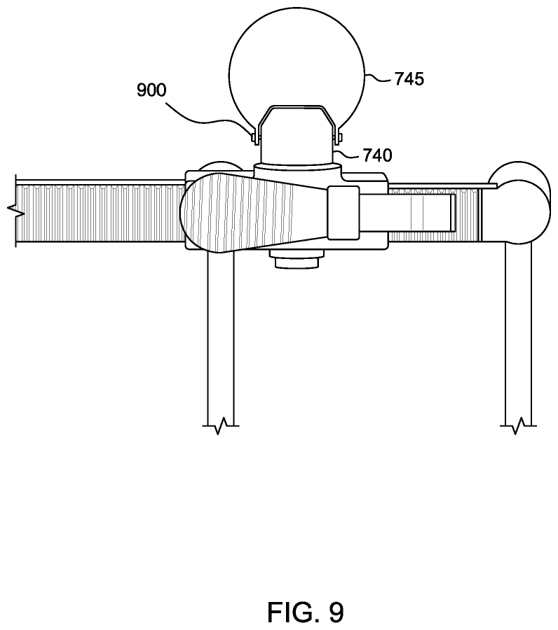

FIG. 9 is an illustration of a rack and pinion-style retractor embodiment having a folding handle 745. The handle 745 is hingedly joined to the knob 740 through hinge 900. The handle 745 has a 90 degree range of motion about the hinge 900 relative to the knob 740. Such a handle 745 allows the surgeon to fold down the handle 745 once the retractor is positioned, thereby reducing the chance that the retractor may be inadvertently bumped or caught by another instrument.

Embodiments of the invention are well suited to implantation of stabilizing devices in non-fusion laminectomy procedures. For example, and without limitation, embodiments are suitable for retracting vertebrae during implantation of the Coflex® or Cofix® interlaminar stabilization device. In general terms, a Coflex® device is implanted through the posterior spine. An incision is made in the patient's back, and the space between the affected vertebrae is prepared by removing bone and ligament tissue to make room for the implant. A spacer is inserted between the vertebrae to estimate whether a proper fit will be attained. When the intervertebral space is prepared, the surgeon taps the Coflex® implant into position and crimps the device around the spinous processes. The foregoing procedure can be modified by using an embodiment of the invention to separate the diseased vertebrae. Bone and ligaments are then removed as usual, and the vertebrae are held in position with the retractor while the surgeon taps the implant into place.

Embodiments are also suitable for use in transforaminal lumbar interbody fusion (TLIF) procedures. Similar to the foregoing non-fusion procedure, the surgeon enters through the back of the spine. The diseased disc is partially removed and an implant is inserted to the interbody space to provide anatomical spacing between vertebrae and facilitate interbody fusion. Bone from the patient's pelvis, allograft bone, polyether ether ketone (PEEK), or titanium are utilized as implants. The implant is inserted to the interbody space, therefore facilitating fusion of vertebrae. Pedical screws and rods are affixed to the back of the vertebrae to provide stabilization. Bone is also grafted to the hardware, forming a bone bridge that stabilizes the vertebrae. The foregoing procedure can be improved by using an embodiment of the invention to retract and hold the vertebrae in position while the spacer and hardware are implanted.

An embodiment of the invention is a spinous process oppositional or longitudinal retractor called the Carr Oppositional Retractor or “C.O.Retractor”. The embodiment is specifically designed to be utilized during the implantation of nonfusion interlaminar procedures such as the Coflex® or Cofix® Interlaminar Stabilization devices. The embodiment is also designed for use in placement of lumbar interbody fusion devices as seen in a transforaminal lumbar interbody fusion (TLIF) procedure.

Coflex® and Cofix® are titanium implants surgically placed in the intralaminar segments of the lumbar spine to treat moderate to severe spinal stenosis. These implants are simple in concept, strong, and flexible enough to mimic normal spine biomechanics and thus “restore” normal movement versus fusion instrumentation designed to “restrict” normal movement.

TLIF implant devices are designed to facilitate lumbar interbody fusion. In order to implant nonfusion interlaminar devices or TLIF implants, a posterior approach to the spine through the skin, posterior lumbar fascia and muscular attachments is performed. Once direct visualization of the posterior spine is achieved, removal of the interspinous ligament and appropriate portions of the laminae allows the placement of C.O.Retractor.

Utilizing a longitudinal rack and pinion type oppositional retractor, the C.O.Retractor generally includes a pair of arms that are opposite to each other. At the end of the arms of retraction, there are two downward 90 degree arms of 25 mm to 100 mm in length. Attached to the inferiorly directed arms are the C.O.Retractor U-shaped blades, as described in more detail supra. These blades dock to the spinous processes of the patient and may be sized to fit both men and women of all shapes and sizes. The particular design of the C.O.Retractor U-shaped oppositional blades attached to the inferiorly angled 90 degree arms places the arms on the opposite side of the spinous process away from the surgeon. This allows better visualization for the surgeon working in the microscope as well as improved longitudinal tissue retraction.

The subsequent longitudinal retraction of the spinous processes further exposes the interlaminar space and makes the ligamentum flavum taut. The improved interlaminar visualization and tension of the ligamentum flavum allows safer and easier surgical removal of compressive tissues.

The C.O.Retractor also greatly facilitates placement of lumbar interbody fusion devices as seen in a transforaminal lumbar interbody fusion or (TLIF) procedure.

Prior to placing the C.O.Retractor, the “Method of Insertion” of the retractor comprises preparation of the spinous processes to optimize the docking of the retractor. The preparation of the spinous processes to accept the C.O.Retractor will allow interlaminar devices such as the Coflex® or Cofix® to be implanted more easily at the end of the surgery.

The preparation of the spinous process to accept C.O.Retractor will decrease the surgical time as it improves visualization. The retractor U-Shaped blades are also the same size as the Coflex® or Cofix® implants so no further carpentry is required.

The C.O.Retractor is specifically designed for both nonfusion interlaminar devices such as Coflex® or Cofix®, as well as TLIF interbody fusion surgeries.

The C.O.Retractor is beneficial to all surgeons who perform laminectomy, nonfusion interlaminar surgeries and TLIF surgeries as it improves the direct visualization of the neural compressive elements that need to be removed. The C.O.Retractor decreases surgical time as well as time under anesthesia for patients, thus directly improving surgical outcomes for patients.

It will be apparent to those skilled in the art that the above methods and apparatuses may be changed or modified without departing from the general scope of the invention. The invention is intended to include all such modifications and alterations insofar as they come within the scope of the appended claims or the equivalents thereof.

Having thus described the invention, it is now claimed:

1. A retractor blade (115), comprising:
a straight first leg (102) of the retractor blade having an end (104) joined to an end (118) of a shank (112), wherein the straight first leg is from zero cm to 6.0 cm in length, and wherein the shank is fixedly joined to a shank spreader;
a straight second leg (106) of the retractor blade having a free end (108), the straight second leg (106) oriented parallel to the straight first leg (102), wherein the straight second leg is from zero cm to 6.0 cm in length; and
a transition (114) from the straight first leg (102) to the straight second leg (106), wherein a width (w) between the straight first leg (102) and the straight second leg (106) is operable to receive a spinous process.

2. The retractor blade of claim 1, wherein the end (104) of the straight first leg (102) is joined to the end (118) of the shank (112) so that a face (113) of the shank (112) does not extend beyond an inside face (103i) of the straight first leg (102).

3. The retractor blade of claim 1, wherein the transition (114) is arcuate.

4. The retractor blade of claim 1, wherein a length of the straight second leg (106) is determined by a length (L₁) of the blade exceeding a distance between a tangent line (T) of an apex (116) of the transition (114) and a parallel line drawn through an end of the straight second leg (106) most proximal to the tangent line (T) and an end of the straight first leg (102) most proximal to the tangent line (T), wherein the length of the straight second leg (106) does not exceed 6.0 cm.

5. The retractor blade of claim 1, wherein the blade comprises a band (400) having a height (h) from about 4.0 cm to about 0.3 cm+/−10%.

6. The retractor blade of claim 1, wherein the first straight leg (102) and the straight second leg (108) are spaced apart by the width (w) from about 0.3 cm to about 2.0 cm+/−10%.

7. The retractor blade of claim 1, wherein the free end (108) of the straight second leg (106) is arcuate and defines an apex (402).

8. A retractor blade, comprising:
a straight first leg (102) of the retractor blade having an end (104) joined to an end (118) of a shank (112), wherein the shank is fixedly joined to a shank spreader; and
a transition (114) arcuately extending from the straight first leg (102) from about 90° to 180° and terminating in a free end (108), wherein a distance between the straight first leg (102) and the free end (108) is suitable for receiving a spinous process.

9. The retractor blade of claim 8, wherein the end (104) of the straight first leg (102) is joined to a face (113) of the end (118) of the shank (112).

10. The retractor blade of claim 8, wherein the transition (114) is arcuate.

11. The retractor blade of claim 8, wherein the transition (114) is a circular segment having a radius between about 1.0 cm and about 0.15 cm+/−10%.

12. The retractor blade of claim 8, wherein the blade comprises a band (400) having a height (h) from about 4.0 cm to about 0.3 cm+/−10%.

13. The retractor blade of claim 9, wherein the free end (108) is arcuate and defines an apex (402).

14. A spinal retractor, comprising:
a first blade (115f) comprising:
a straight first leg (102f) having an end (104f) joined to an end (118f) of a first shank (112f);
a straight second leg (106f) having a free end (108f), the straight second leg (106f) oriented parallel to the straight first leg (102f); and
a transition (114f) from the straight first leg (102f) to the straight second leg (106f), wherein a distance between the straight first leg (102f) and the straight second leg (106f) is suitable for receiving a spinous process;
a second shank (112s) opposing the first shank (112f);
a shank spreader (501) in spreadable communication with the first shank (112f) and the second shank (112s); and
a ratchet (524) in ratcheting communication with the shank spreader (501).

15. The spinal retractor of claim 14, further comprising:
a second blade (115s) comprising:
a straight first leg (102s) having an end (104s) joined to an end (118s) of a second shank (112s);
a straight second leg (106s) having a free end (108s), the straight second leg (106s) oriented parallel to the straight first leg (102s); and
a transition (114s) from the straight first leg (102s) to the straight second leg (106s), wherein a distance between the straight first leg (102s) and the straight second leg (106s) is suitable for receiving a spinous process.

16. The spinal retractor of claim 14, wherein the shank spreader (701) comprises:
a rack (730) fixedly joined to the first shank (112f);
a carrier (735) fixedly joined to the second shank (112s), the carrier (735) slideably engaging the rack (730) through a pinion gear;
a knob (740) rotatably communicating with the pinion gear;
wherein the ratchet (724) is mounted to the carrier (735) and comprises a spring-loaded pawl (750) biased toward engagement of the rack (730), the pawl being configured to allow the carrier (735) to spread the first shank (112f) and the second shank (112s) while engaging the rack (730).

17. The spinal retractor of claim 14, wherein the shank spreader comprises:
a first member (510f) having a first handle (514f) and the first shank (112f), and having a first pivot joint (610f) between the first shank and the first handle; and
a second member (510s) having a second handle (514s) and a second shank (112s), and having a second pivot joint (610s) between the second handle (514s) and the second shank (112s).

18. The spinal retractor of claim 14, wherein the first shank and the second shank each include a 90° turn.

19. The spinal retractor of claim 16, wherein a handle (745) is linked to the knob (745) through a hinge (900).

20. The spinal retractor of claim 19, wherein the handle (745) has a 90° range of motion about the hinge (900) relative to the knob (740).

21. A method of vertebral retraction comprising the steps of:
providing a first vertebra and a second vertebra adjacent to the first vertebra;
providing a spinal retractor, comprising:
a first blade (115f) comprising:
a straight first leg (102f) having an end (104f) joined to an end (118f) of a first shank (112f);
a straight second leg (106f) having a free end (108f), the straight second leg (106f) oriented parallel to the straight first leg (102f); and
a transition (114f) from the straight first leg (102f) to the straight second leg (106f), wherein a distance between the straight first leg (102f) and the straight second leg (106f) is suitable for receiving a spinous process;
a second shank (112s) opposing the first shank (112f);
a shank spreader (501) in spreadable communication with the first shank (112f) and the second shank (112s); and
a ratchet (524) in ratcheting communication with the shank spreader (501);
inserting the first blade between the first vertebra and the second vertebra;
actuating the shank spreader (501), causing the first blade to impinge a spinous process of the first vertebra; and
separating the first vertebra from the second vertebra by a predetermined amount.
